(19) United States
(12) Patent Application Publication
(10) Pub. No.: US 2022/0152510 A1
(43) Pub. Date: May 19, 2022

(54) Gameplay Evaluation Method and System

(71) Applicant: Gifted Bill Enterprises, LLC, Marietta, GA (US)

(72) Inventors: Sreekanth Sunil Thankamushy, Porter Ranch, CA (US); William H. Allen, Marietta, GA (US)

(21) Appl. No.: 17/526,686

(22) Filed: Nov. 15, 2021

Related U.S. Application Data
(60) Provisional application No. 63/113,548, filed on Nov. 13, 2020.

(51) Int. Cl. A63F 13/60; A63F 13/77; A63F 13/35; G09B 5/04; A61M 21/00

(52) U.S. Cl. CPC A63F 13/60; A63F 13/77; A63F 13/35; A61M 2021/0027; A61M 21/00; A63F 2300/6009; A63F 2300/552; G09B 5/04

(57) ABSTRACT

A gameplay evaluation method and system are described. Tags may be embedded in game software which, when triggered by gameplay events, transmit gameplay information to a server or database. A computer may retrieve the information stored on the database and an analyzer may analyze the information. A developer may use the results from the analyzer to improve the game or fix bugs in the game code.

### CROSS-REFERENCE TO RELATED APPLICATIONS

This application claims priority to U.S. Provisional Application No. 63/113,548, filed on Nov. 13, 2020, which is relied upon and incorporated herein in its entirety by reference.

### TECHNICAL FIELD

This disclosure relates to gameplay software and its evaluation.

### BACKGROUND

When a developer releases a video or software game, the developer typically has no way to evaluate how the game is being played by the public. The kind of feedback mechanisms and analytics that do exist currently are limited to broad picture items such as downloads, replay counts, user attrition rates, traffic data of particular parts of the game etc. What is missing is actual gameplay data, which is to say, a way to understand exactly how the public is interacting with the game itself, in all its complexity. It would be beneficial to the developer to know where the players are enjoying the game more, where they are getting frustrated etc.

While it is possible for the developers to elicit feedback from users, such methods are not real-time deliverables, and require additional resources from the developer. Further, only some gamers may respond, which might not give a complete picture of the true play of the game.

Along the same lines, developers are looking for ways to address situations where users of the program perform more poorly not due to game design, but the state of the player at that point in the game. Upset and unfocused players can lead to poor game play experiences, which can lead to a drop in effectiveness of the game, especially in educational-based games.

Therefore, there is a need for a system and method that addresses the shortcomings discussed above.

### SUMMARY

It is to be understood that this summary is not an extensive overview of the disclosure. This summary is exemplary and not restrictive and it is intended to neither identify key or critical elements of the disclosure nor delineate the scope thereof. The sole purpose of this summary is to explain and exemplify certain concepts of the disclosure as an introduction to the following complete and extensive detailed description.

The present disclosure relates to a system and method for gameplay evaluation. In an aspect, the invention is directed at a gameplay evaluating method (GEM) and system. The GEM system allows a game developer to observe in real-time how players worldwide are interacting with the game. The GEM system allows the developer to keep a birds-eye view of gameplay as it happens and observe patterns of gameplay behavior of thousands of live players. Therefore, the GEM system is a tool that tells the developer which parts of the game are working well and which are not working well.

Examples of what the developer can observe may include: which activities are more users engaged with, how long a particular activity is used by a user, the order in which activities are done, places that have technical issues that prevent playing, when players are collecting points and rewards, what levels they are in at the moment and the like.

Part of the GEM system may include a focus improving mechanism to improve player attention by allowing the game to intervene when it detects that a user appears to be having difficulty or a certain task is taking longer than anticipated. In an aspect, the focus improving mechanism can be called a Bindu Focus, and implement a method for helping a user focus on the task at hand. Such a focus method may comprise having a character guide give voice instructions to the player which is then accompanied by clicking on a Mind's Eye icon.

In an aspect, the invention is directed at a system for evaluating gameplay of an interactive program, the system including a server that places game tags within the code of the interactive program, receive data captured by the game tags from a user device using the interactive program; and stores and evaluates the data captured from the game tags to evaluate and improve the interactive program. In some instances, the server includes a game tag manager that allows a developer to place the game tags with game play of the interactive program. The game tags can include special tag lines of code configured to pull data generated during use of the interactive program on the user device when triggered. The pulled data can include a location of a game tag, a game ID, and a broadcast. The system can communicate with several different user devices to evaluate the interactive program from the data collected from each of the user devices. In some aspects, the system can evaluate different interactive programs at the same time, and each of the interactive programs. The server is configured to receive the data captured by the game tags in real time or at planned timed-intervals.

The system can also include an analyzer program that applies questions to filter the data captured in order to further evaluate the interactive program. This can be done without identifying the individual users of the interactive program. In some aspects, the analyzer program can work with a reporting module configured to report back performance within the interactive program of a user of the user device based upon the evaluation of the data captured from the game tags.

In some instances, the system includes a focus improving mechanism that is presented on the user device when the evaluation of the data captured from the game tags indicates a lack of focus. The focus improving mechanism can provide audio instructions and video instructions to the user.

In an aspect, the invention is directed at a system for evaluating interaction of a user with an interactive program that includes a sever with communication means, a game tag manager configured to place game tags at events of gameplay of the interactive program, a database, an analyzer program, and a reporting module. The system is configured to interact with numerous user devices on which the interactive program operates. The game tags are configured to pull gameplay data captured during a user's interactions with the interactive program on the user device when the game tag is triggered. The data captured can include a program identification, game tag location, and broadcast. The data captured would be pulled when the game tags were triggered, and then would be communicated with the server. Upon receiving the data, the server is configured to store the data captured by the game tags in the database, provide questions, via the analyzer program, to assist in filtering the captured data to analyze the captured data to evaluate effective of the interactive program, evaluate, via the analyzer program, an individual's performance, and report, via the reporting module, the individual's performance.

The GEM system is valuable because when a developer releases a game, typically a mobile game, he has no idea how the game is being played. The data generated by the GEM system allows the developer to test his assumptions on his audience, and lets him make appropriate changes to improve the game, in subsequent updates. Further, it also assists players in tackling difficult areas in gameplay in a focused manner.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 illustrates a schematic representation of a gameplay evaluation system according to an aspect of the present invention.

FIG. 2 is a schematic representation of the GEM server according to an aspect of the present invention.

FIG. 3 illustrates a game tag manager according to an aspect of the present invention.

FIG. 4 is a schematic representation of an interactive program with game tags within the gameplay according to an aspect of the present invention.

FIG. 5 illustrates a list of filter questions used by an analyzer according to an aspect of the present invention.

FIG. 6 illustrates screen shots of a graphical user interface (GUI) of the analyzer according to aspects of the present invention.

FIGS. 7-11 illustrate aspects of the focus improving mechanism according to aspects of the present invention.

FIG. 12 is a schematic representation of computing devices utilized by the GEM system according to an aspect of the present invention.

### DETAILED DESCRIPTION

The present disclosure can be understood more readily by reference to the following detailed description, examples, drawings, and claims, and their previous and following description. However, before the present devices, systems, and/or methods are disclosed and described, it is to be understood that this disclosure is not limited to the specific devices, systems, and/or methods disclosed unless otherwise specified, as such can, of course, vary. It is also to be understood that the terminology used herein is for the purpose of describing particular aspects only and is not intended to be limiting.

The current invention is directed to a gameplay evaluation method and system that is configured to allow a game developer to observe and monitor in real-time how players worldwide are interacting with a video/software based game. In an aspect, the invention is directed at a gameplay evaluation method (GEM) and system that implements the method. The GEM system 10 can be utilized to evaluate the performance of any given program/software 200 that involves some form of user interaction (e.g., training programs, utility programs, and the like). These interactive programs 200 are operated on user devices 100. In such aspects, the GEM system 10 is configured to be able to communicate with downloaded/disseminated versions of the interactive programs 200 on these user devices 100. For example, the GEM system 10 can be utilized with the interactive program 200. In an aspect, the interactive program 200 can be a mobile game 200 hosted on a mobile device 100 like a tablet or smart phone, or a video game 200 played on a computer or console 100. While the GEM system 10 can be utilized with any interactive program 200 that operates on a user device 100, it is preferable that the program 200 has some type of advancing gameplay or mechanism that advances the user through the program 200. Further, the GEM system 10 is configured to be used with various user devices 100 that have communication means, discussed in more detail below.

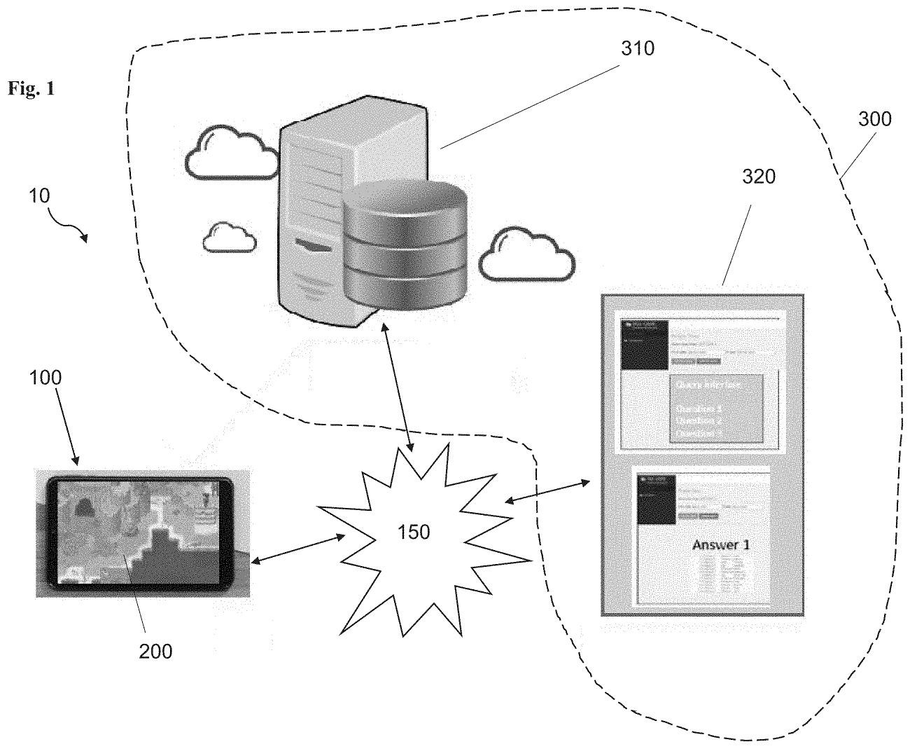

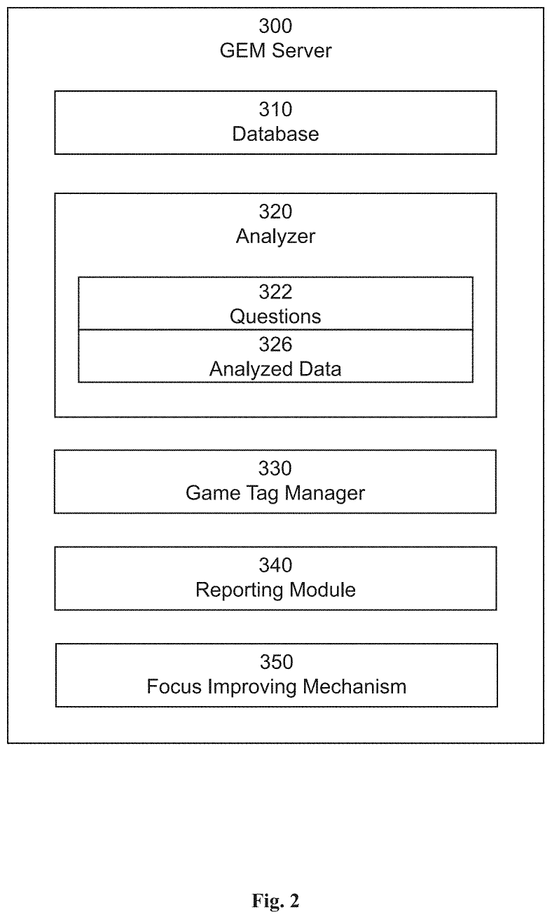

In aspect, the interactive program 200 can take the form of a downloadable program available via various networks (e.g., internet 150) and various application stores (e.g, Google Play, Apple Play, etc.) to be used on various user devices 100. In other aspects, the interactive program 200 can be distributed physically (e.g., via a disk, a portable drive, or preloaded). In an aspect, as shown in FIGS. 1 and 2, the GEM system 10 works with a user device 100 that hosts an interactive program 200 (e.g., a downloaded game on a tablet or smart phone, or a game loaded from a disc or cartridge for use on a PC or console), with the user device 100 configured to be able to communicate to a GEM server 300 (see FIGS. 1 and 2). In an aspect, the GEM server 300 includes a database 310, an analyzer 320, a game tag manager 330, a reporting module 340, and a focus improving mechanism 350. The database 310 is configured to store captured data from the activities of the user with the interactive program 200, and the analyzer 320 is configured to analyze the collected data. These and other aspects are discussed in more detail below.

In any aspect, the GEM system 10 has the ability to collect interaction data from the interactive program 200 based upon user interaction for analysis. Further, the GEM system 10 is configured to communicate with several user devices 100 on which the interactive programs 200 are used. In such aspects, the user devices 100 can be any device on which an interactive program 200 can operate and has the ability to communicate to other devices via a network. In another aspect, the GEM system 10 can be configured to work with several different interactive programs 200 at the same time. In such aspects, all that is needed is a way to clearly identify the different interactive programs 200 from one another (e.g., a program ID). In an aspect, the collected data can be transmitted in real time to the GEM server 300 over a network 150 for storage (database 310) and analysis (analyzer 320). In other aspects, the GEM system 10 can be configured to collect the data locally (save in file on the user device 100) which can be downloaded and analyzed later.

In an aspect, the server 300 of the GEM system 10 can be affiliated with the developer of the interactive program 200. For example, the server 300 can be that of the developer of the interactive program 200. In other aspects, the server 300 can be configured to act as an intermediate with a separate server operated and controlled by the developer. Therefore, references to a server 300 herein cover both functionalities.

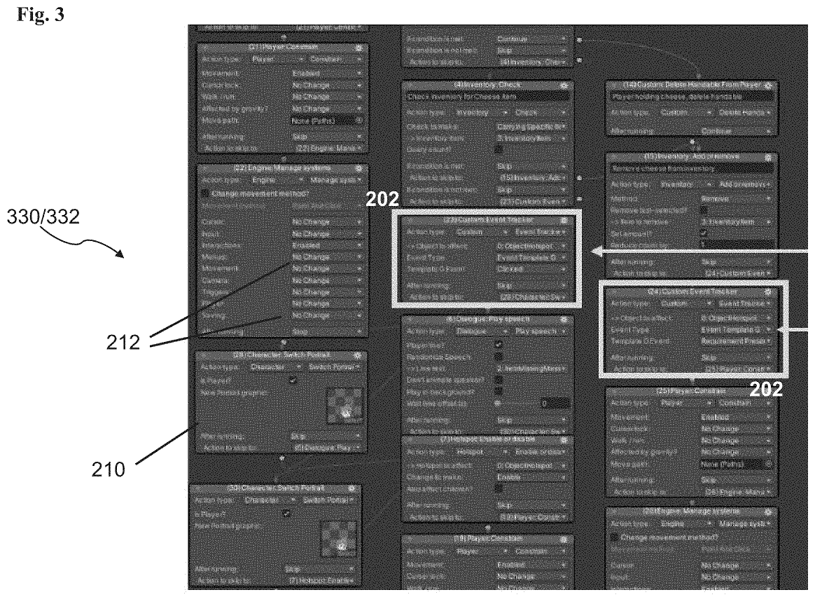

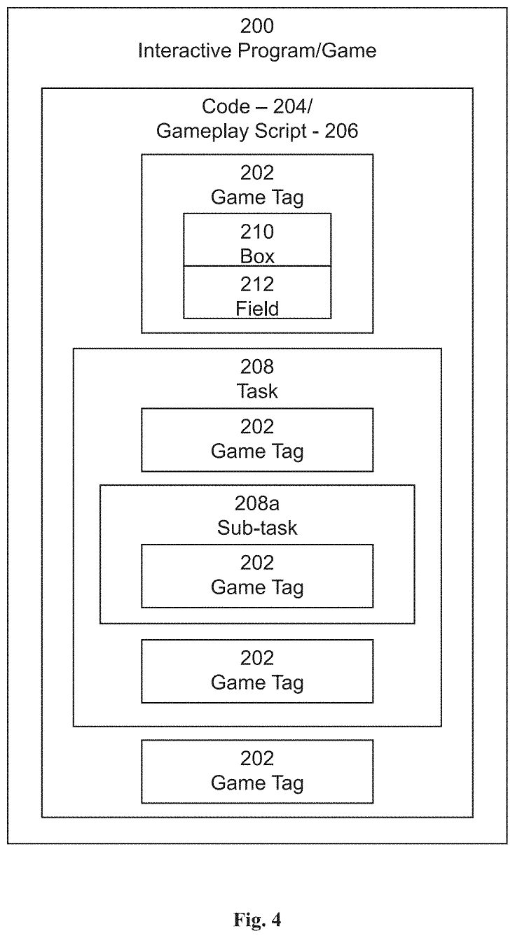

In an aspect, illustrated in FIGS. 3-4, the GEM system 10 implants game tags 202 placed within code 204 of the program 200 at various parts of the gameplay/script/game flow 206. Within this application, there is a distinction between game code 202 and game flow/game play/script. The game code 204 is the underlying code that makes the game work. Game flow/gameplay/script refers to the sequence in which game events happen (i.e., A happens, then B happens, then C happens; player walks to the shelf, picks up some fruit, then eats the fruit, then walks to his room etc.) The game code 204 makes up the gameplay 206.

The game tags 202 are snippets of code/short strings that serve as markers within the code 204 of the gameplay 206. The game tags 202 are placed in the game/interactive program 200 by the game developer at any point of interest in the game flow 206. This is done by inserting special ‘tag’ lines of code 202 directly in the general code 204 of the game, or by adding tag nodes 202 (see the boxes in FIG. 3), or fields within existing nodes (i.e., pockets of information game designers string together, to set up events in the game). When the tags 202 are activated, the game tags 202 will then send signal back to the GEM server 300 above the event that has occurred (e.g., when player has completed all the objective). Every game tag 202 placed throughout the game sends a signal when that event has been achieved.

The tags 202 can be placed at the point in the gameplay script 206 where the player reaches a certain activity, or when the player completes a certain activity, or when the player enters or exits a certain map etc. FIGS. 3-4 illustrate examples of several event tags 202 placed within program code 204 of the gameplay script 206 according to an aspect of the present invention. In an aspect, the game tags 202 can be placed at various places within the gameplay 206. These game tags 202 can be placed within the gameplay/script 206 wherever the game developer chooses. However, the tags 202 are typically placed at known events within the gameplay 206 (e.g., the beginning or ending of a task (e.g., completing a puzzle) that needs to be completed). In addition, the gameplay 206 can include various tasks 208 that the player should complete. The tags 202 can be placed before or after tasks 208. The game tags 202 can also be placed at various sublevels/subtasks 208a of a task 208 (e.g., a certain puzzle includes 4 subparts, the game tags 202 can be placed at the beginning and end of each subpart 208a, as well as at the beginning and ending of the overall puzzle 208). Further, the game tags 202 can be placed when various events occur, or milestones, within the gameplay 206. In an aspect, the GEM system 10 considers events as notable things that happen in the game (e.g., Player collected an apple, player found the key to open a door, the door got opened etc.). The GEM system 10 can also classify critical events as milestones—events that are of particular importance (Player completed a level, Player achieved all objectives in a level, etc.). The distinction can be utilized to assist in evaluating the overall performance of the game, allowing the developers to create a hierarchy of activities, and easily identify portions within the game script 206 for the GEM system 10 to place game tags 202 to capture the data, discussed below.

In an aspect, as shown in FIG. 3, a game tag manager 330, via a graphical user interface 332, provides a game tag 202. In an aspect, the game tag 202 can look like a box 210 with various fields 212 for the developer to provide input. The GUI 332 of the game tag manager 330 may be employed by the developer to arrange the game tags 202 at appropriate points in the script 206 (e.g., at milestone events). This box 210, after set up, contains/identifies all the information that needs to be sent to the GEM server 300. In other words, the fields 212 identify the type of data will be collected by the game tag 202 from a user's interactions with the interactive program 200 when the user reaches the section of the gameplay 206 in which the game tag 202 is located. The fields 212 of the game tags 202 can include, but are not limited to, the following information: identification of the interactive program 200 itself, location of the game tag 202 within the script/gameplay 206 (and placed within the code 204), type of situation that should activate/trip the tag (e.g., a player in the vicinity (these can be predetermined, or the developer defines them)), and the type of signal that should be broadcast within the gameplay 206 when tripped/triggered. In other words, if a player accomplishes a task/event within the gameplay 206, and that event triggers some additional event (e.g., a reward), the tag will send back to the GEM sever 300 the particular triggering event and the type of reward that is to be given. For example, if the player reaches the red gate after accomplishing his tasks, this signal is broadcast to the rewards systems of the game that generates rewards for the player at the gate. Examples of the types of situations for tripping the game tag 202 include, but are not limited to, the following: Activity Started, Activity reached halfway, Activity completed, Point A reached in map, Point B reached in map, Map exited, Item A collected, Item A destroyed, 25% of points gained, 100% of points gained, Points low, and the like. In other words, the situation types are infinite, and are determined by the gameplay 204 of the program 200.

In an aspect, the GUI 332 of the game tag manager 330 of the GEM system 10 allows a designer to place the game tags 202 within the gameplay 206 of the program code 204 during programing or after programing according to an aspect of the present invention. The game tags 202 can be entered into code 204 directly, or through nodes (see FIG. 3), or via an API. In such aspects, the game tag manager 330 is used for the game developer to plug in the necessary game tags 202 wherever he feels is important. In another aspect, the game tag manager 330 will also place a conversion script which converts the information inputted into necessary instructions that the game 200 executes for the sending of the tag 202, and more specifically the information captured by the tag 202 and sent to the server 300.

The GEM system 10 can be used with a program 200 even after the game has been developed, as long as the developer can go back to the code 204 and retroactively implement it, though it is best done during game development. Since the game tags 202 necessary for various games are dependent on the particular game, custom game tags 202 can be created to suit the game. In some aspects, the GEM system 10 utilizes the GUI 332 of the game tag manager 330, where a game developer can input text, and generate all the game tags 202 that are necessary for their particular game at the onset through a simple user interface, before proceeding to place the game tags 202 within the game code 204 of the gameplay 206. In another aspect, APIs (e.g., set of simple lines of code that allows other developers to retrieve the GEM functionalities) can be used for this, so that any game developer can access the libraries and processes to generate the game tags 202.

During gameplay, when the player reaches or activates tagged points thereby indicating the player's current status in the game or the interactive program 200, a game tag 202 is tripped. Tripped means that a particular tag 202 has been triggered. The triggering happens in the game 200, during gameplay when the player completes what the game tag 202 is checking for. When it is triggered, a small text string (e.g. “Making Tea Activity Initiated” “Making Tea Activity 50% completed” “Making Tea Activity Exited” etc.) may be transmitted to a remote server 300.

When a tag 202 is tripped, the information is sent from the game 200, via a network 150 (e.g., the internet), to the GEM server 300. The information sent can include the tag 202 and the current time stamp. For example, a user can be playing a game 200 on a mobile tablet 100 or a PC 100, and the tag 202 and time stamp are transmitted via the internet 150. In an aspect, this is done instantly. In other aspects, that data can be sent periodically only—say every 1 minute or so. In other aspects, as discussed elsewhere, the game tag information can be stored for later uploading and analysis with the data being stored on the user device 100 until called to be sent out.

In an aspect, for improving overall gameplay of the program 200, names or identifiable data of players are not tracked by the GEM system 10, which therefore keeps identities protected. In another aspect, for use with the reporting module 340 (discussed in more detail below) the GEM system 10 can track an individual performance using a Device ID associated with the user device 100. In an aspect, the Device ID can be randomly generated number by GEM system 10, the manufacturer of the user device 100, or by the distributing platform of the program 200 (e.g., by Apple or Google when an app is downloaded into a device from their app stores). In such aspects, a user of the program/game 200 can create a profile to play the game 200, which information can also be included and tracked by GEM system 10. In an aspect, profile names are pre-made, and are not customized by the player. For example, the GEM system 10 may implement profile names as names of fruits and vegetables (e.g. apple, orange, carrot, celery etc.). In other aspects, profile information, provided by the user of the program 200, can be used as well. In an aspect, the GEM system 10 may also track location information of the device 100 (e.g., the city name that the app was downloaded to). This data can be collected by the device 100 operating the game 200, or from the distributor (e.g., Google and Apple). So, in some aspects, a combination of the city ID, device ID, and the profile name is sufficient of an identifier to denote a particular user in a particular part of the world. This is useful for macro data of the game. This is a piece of information that can be used for the following: where in the world are players playing the game; what countries or cities seem to like the game more; what cities do not seem to like this particular feature of the game; should advertising increase in that city?

The game tags 202, when tripped, are configured to capture and transmit any data that can be generated from the user's gameplay/activity within the program. This data can include player status (e.g., health, experience level, points earned, attributes gained), player progress (stage of the game, percentage completed, events completed, tasks completed), and the like. Below are some examples of events and the data generated when a game tag 202 is tripped.:

The moment a new map is entered by the player. [Game event: Map Activity; Name of Map: Forest. Status: Entered; Timestamp: 2021-01-19 03:14:07]

The moment a player exists a new map. [Game event: Map Activity; Name of Map: Forest. Status: Exited; Timestamp: 2021-01-19 03:14:07]

The moment a collectible has been picked up by the player [Game event: Name of Map: Forest; Activity: Collectible pickup; Name of Collectible: Blue Shell; Status: Collected; Timestamp: 2021-01-19 03:14:07]

In an aspect, tags 202 can also be automatic. The developer can choose the game to relay situations of the player at regular intervals (as in the state of the player below). In addition, periodic broadcasting of the health state of the player tags can be utilized, producing the following event. [Game event: Health status; Current health: 25%; Timestamp: 2021-01-19 03:14:07]

Tags can also be set up to relay when particular situations occur in the game. In the example below, the trigger is tripped when the game detects that the player has been stuck/frozen/not responsive: Mobility state of the player [Game event: Mobility; Mobility state: Immobile; Time: 3.34 minutes; Timestamp: 2021-01-19 03:14:07]

The above are examples of the data collected, and are no way mean to limit the data transmitted. The game tags 202 can capture and report any data that is associated with a user's progress in the program 200.

Once generated, the information collected by the game tag 202 can then be transmitted from the user device 100 to the server 300 via the internet 150. In an aspect, the game tags 202 are then stored in a real time database 310 (See FIGS. 1-2). In an aspect, various databases 310 can be utilized. For example, NoSQL, MySQL and other known types of databases may be utilized by the GEM system 10. In an aspect, the tags 202, their corresponding time-stamps, and the information captured from the tag, are saved. As players play, thousands or hundreds of thousands of these entries build up in the database 310 of the GEM server 300. As discussed above, captured data in the database 310 includes, but is not limited to, location/city ID, device ID, time stamp, game activity information (maps, activities, and any other game event of interest to the designer).

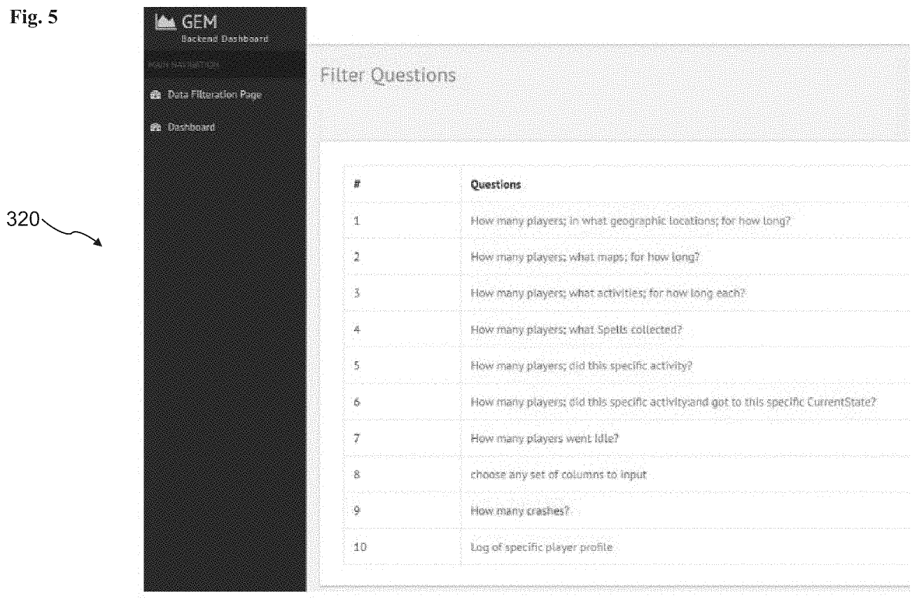

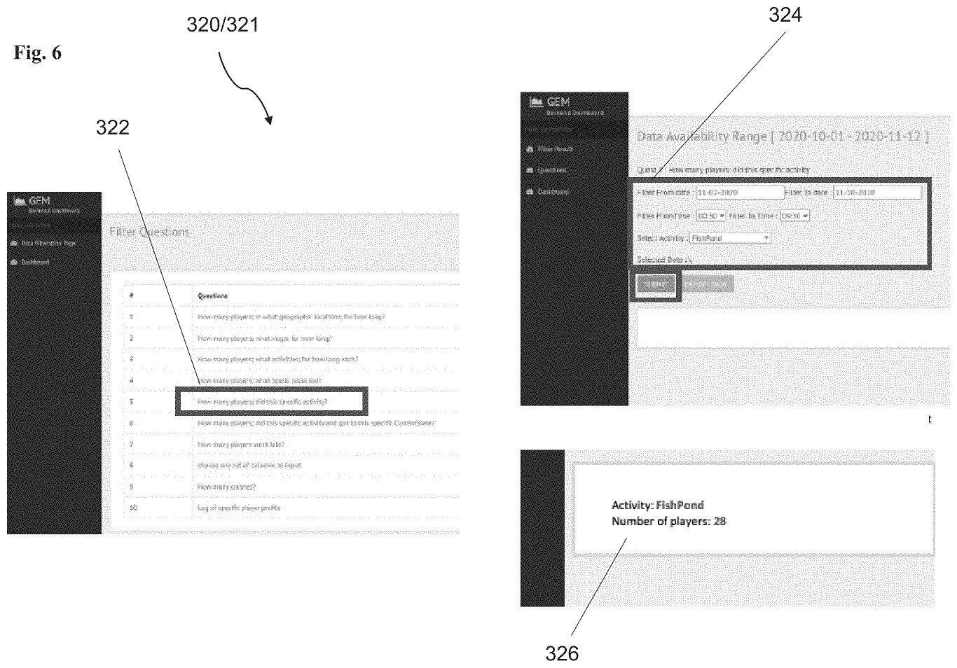

The GEM system 10 can then analyze the data as collected. In an aspect, the GEM system 10 includes an analyzer program 320. In an aspect, a standard query list used to sort and filter the data collected for the analyzer 320 can be viewed in an analyzer associated GUI 321, as shown in FIGS. 5-6. These questions are the most typical type of data a developer would want to gather. For example, some standard queries can include, but are not limited to, those shown in the exemplary dashboard shown in FIG. 5 (e.g., how many levels did the players complete, what is the current player level, geographic location of most players, etc.). The GEM analyzer 320 evaluates the data gathered from the database 310 and runs it through algorithms (see below), to arrive at the answers that are displayed to the game developer (See FIG. 6). Over time, dozens of these questions can be created and algorithms written to analyze the data and produce the answers. Ten such exemplary questions are illustrated in FIG. 6. In an aspect, developers will be provided with specific questions and algorithms to gain specific insights of particular products. The list of questions can include standard questions, or allow the developer to input queries specific to the interactive program 200. These questions are solved using algorithms suitable for each of the problems (e.g., specific to the question that is being asked). Below are such examples of the queries:

Examples of queries, and the algorithm used to get the answers:

How many levels did the players complete?
Display the level tags received in the server
Count the number of level tags, and display the value
   What is the current level the players are at?
Get the last level that was time-stamped.
   How many activities did the players complete?
Select and Sort the activities with the states tagged, “Completed”
Display the number
   How long did activity X take to complete?
For activity X, sort the state tags, “Started”, and “Completed”
Measure the time between the time-stamps, and display the value.
   How many places did the player get stuck at?
Measure the amount of time player has spent idling.
If time spent is more than X, flag this as a potential problem area.
If the player turned off the device, subtract that player ID from the subset (this is to make sure we do not unnecessarily flag users that log off)
Display all these problem areas.
   How many points does the player have currently?
Send automatically to the GEM server the points value of the player at the end of each activity.
Display the points of the player at the end of each level
Highlight the most current points marked at the very last activity
   Present what the most popular activities done by players between Monday and Thursday.
List all activities done by all players between Monday and Thursday
From this list, isolate the activities completed by over 60% of the players
From this isolated list, further isolate activities done more than 2 times.
Present this list

The above questions are merely examples of questions that the GEM system 10, via the analyzer 320, can use to assist in reviewing and generating useful data for a developer. Depending on the specific needs of specific game developers, data can be mined and customized to their special needs. In an aspect, AI and machine learning can be utilized to review the code 204 and gameplay 206 of the interactive program 200 to analyze the events and determine what type of questions and data should be requested in game tags 202. Various other questions that would be useful for the analysis of data resulting from the gameplay of a program can be utilized. In other words, there are no limits on the questions that the analyzer 320 can use. In an aspect, developers utilizing the GEM system 10 can create and implement their own questions, selecting the desired data they want analyzed and how they want such data to be analyzed. Questions can also be asked about large population-based behaviors. Example:
In all of North America, how many players have discovered the Garden map?
Gather all the player IDs of players in cities within North America into a group
Check the number of players within this group who have entered Garden map, and display it

As shown in FIG. 6, an analyzer 320 can help a developer determine which data to evaluate. The developer can select a questions 322, via the interface 321 (the analyzer GUI in this example), from the analyzer 320. At the analysis tab 324 of the analyzer 320, the developer can then further filter the data to be analyzed (data range, activity, time range, etc.). For example, a broad query might be, “How many players in the west coast of the US have completed the game till Map 5?”—The data can then be filtered further with a query, “out of these, how many completed the game till Map 5, within the past one week?” Various other parameters can also be selected to aid the developer. From here, the analyzer 320 can then generate the selected analyzed data 326 (or response) which can answer the developer's question. The analyzed data 326 can then be reviewed by the developer to determine various aspects of user interaction.

The analyzed data 326, once gathered and selected, can be used in various ways, including in game development. The analyzed data 326 can be reviewed by the developers to determine what areas of the game should be improved. For example, if the developers find that many players do not do activity X in sufficient numbers, there could be an error (e.g., a bug) with the game code 204 with activity X. The developers can then examine the game/program at activity X, identify the error, and correct it. The game can then be updated almost immediately to check to see how the public responds. If the correction is effective, then the GEM system 10 would report back that a sufficient number of people completing activity X. In some aspects, the AI and machine learning can also suggest corrections for the game play 206 and code 204.

In addition, the analyzed data 326 can be used for user feedback. For example, for educational games directed at children, the GEM system 10 can collect data about a specific user, analyze the data, and send to the parents, via a reporting module 340, to show the child's progress throughout the game. For example, if the game is directed at reading, the GEM system 10 can monitor the child's progression through the game (e.g. Johnny completed 54 out of 60 word activities last week), and report back the results to the parent. The reports are compiled by GEM, to send to (in our case) parents about how their child is doing in the game. Metrics is basically a report that describes scores the player obtained at various game challenges (such as a quiz, a puzzle etc.) So, tags 202 can be utilized to improve the game program itself, as well as to track and evaluate the progress of individuals as they participate with the interactive program.

In some aspects, the GEM system 10, and its components, are able to identify that the poor performance of an individual is not the result of a mistake in the coding 204, but the lack of focus of the individual. In such instances, the GEM system 10 can provide tools to the user to supplement or improve their performance. In an aspect, the GEM system 10 can provide a focus improving mechanism 350 for the user. The focus improving mechanism 350 can provide suggestions and instructions to the user that assist in improving the individual's performance. The focusing improvement mechanism 350 can utilize text, visual/graphical, and audio components, as well as interactive means, to help improve the individual's performance. In an aspect, the focus improving mechanism 350 can be a component of the GEM system 10, or it can be programmed directly into the interactive program 200.

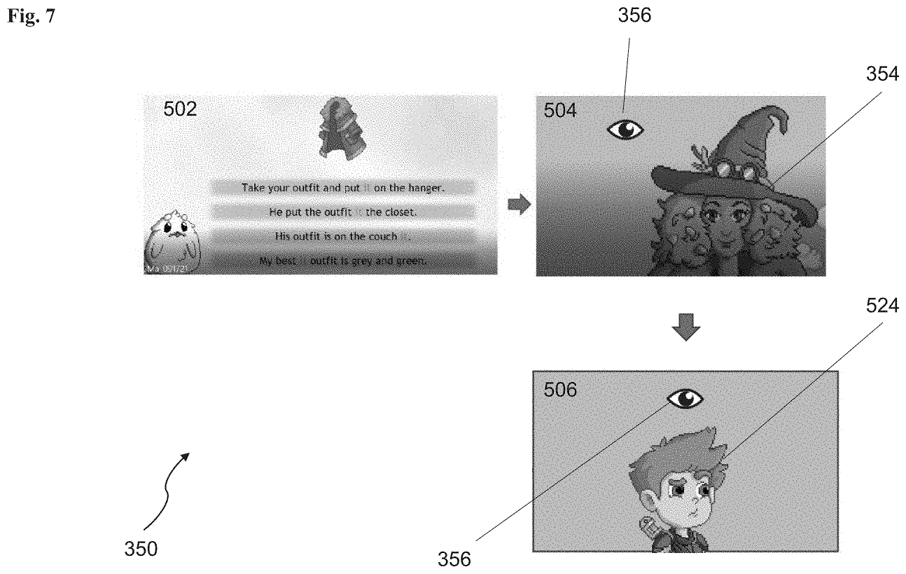

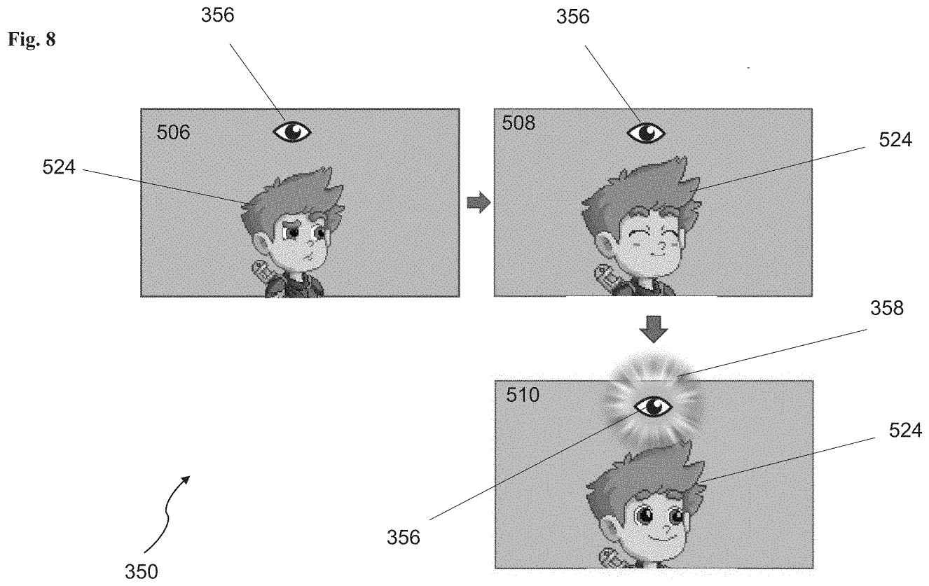

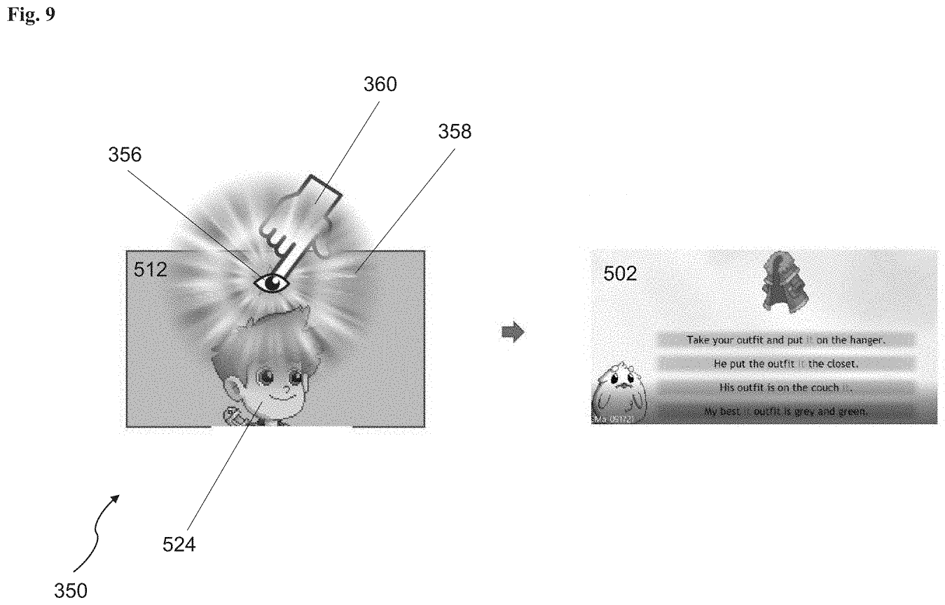

As discussed above, a player may use a video game 200 to supplement or improve their learning of certain skills. For example, The Magical I Am™—Sky Village™ application is an example of such an interactive game 200 which helps improve the learning experiences of children. In this game 200, it is useful to know at which point the player (e.g. a child with dyslexia) has difficulty and may require using techniques to focus their attention. The GEM system 10 can provide the monitoring functionality (via the reporting module 340, which can identify and track individual performance) needed to identify such an occurrence of loss of focus or increased frustration. Upon identifying this time period in game play, the GEM system 10, utilizing the game tags 202 in some instances, can call upon the focus improving mechanism 350. Referring back to the Sky Village application 200, the focus improving mechanism 350, which can include a focal point 352 called a Bindu Focus according to an aspect, within this game, can be utilized, as best described in reference to FIGS. 7-11.

FIGS. 7-9 illustrate several frames 502-512 of a storyline in an educational game 200 according to an aspect. A first frame 502 illustrates an activity in which the player is taking part. The activity depicted may, for example, be an activity where the player must correctly identify certain uses of a word. For example, the user may be asked to determine which sentence uses the word “it” in the correct location. If the player takes longer than a certain threshold of time (e.g., the threshold can be predetermined, or later determined based upon data generated by GEM system 10 via tags 202) to correctly identify the proper answer, then the focus improving mechanism 350 can be activated. As discussed above, the focus improving mechanism 350 can utilize a variety of communication means (textual, visual/graphical, and audio) to help the user regain focus. For example, the focus improving mechanism 350 can produce a player/avatar/character guide 354 to provide instructions/suggestions to the user. For example, the character guide 354 can appear on the screen to give the player advice in a second frame 504. The character guide 534 can provide instructions to help refocus the user, similar to the methods disclosed in U.S. Pat. No. 11,094,218, incorporated in its entirety herein.

For example, the character guide 354 asks the player to “check your Mind's Eye”, with a graphical representation of the Mind's Eye 356 appears on the screen. The Mind's Eye 356 is a focal point on which the user can concentrate. Then the Mind's Eye 356 may be shown above the head of the player avatar 524 as shown in a third frame 506.

Vocal instructions can be given as well. For example, the voice of the character guide 354 may give verbal instructions to the player. For instance, the instructions may include “on my count, take a deep breath” or “breathe in slowly. Hold 1, 2, 3. Now breathe out slowly” or “Now close your eyes” or “Close your eyes and breathe in slowly.” In an aspect, the focus improving mechanism 350 can monitor the actions of the user using components (e.g., camera, microphone, motion sensors) of the user device 100. In other aspects, the focus improving mechanism 350 does not monitor the actions of the user—this is done because in most instances, the user will follow the instructions of the focus improving mechanism 350. Otherwise, the GEM system 10 can be configured to have the user repeat activities in the game 200 until they have reached or surpassed the certain performance threshold that triggered the activation of the focus improving mechanism 350.

Additional effects may occur, such as music or other noises or visual effects, to encourage the player to follow the focus-inducing instructions. For example, the player avatar 524 can be configured to close its eyes in a fourth frame 508 following the advice from the character guide 354 to encourage the player to do the same. In some aspects, a facial recognition algorithm may be utilized to detect when the player viewing the screen closes their eyes, and have the player avatar follow suit, as a way of reinforcing the need to close the eyes to the player. Also, as shown in a fifth frame 510, the Mind's Eye 356 may be surrounded by a glowing aura 358. In other aspects, various other graphics and effects can be employed to assist the player focus.

In addition, the focus improving mechanism 350 can include an interactive component that requires confirmation of the user to perform a certain activity. For example, as shown in a sixth frame 512, the focus improving mechanism 350 may require the player to move a mouse icon (e.g. a pointing finger) 360 onto the Mind's Eye 356 and select/click the Mind's Eyes 356. The required action can trigger additional actions. Additional commands (vocal or text) or visual actions can occur. For example, when the player clicks the Mind's Eye icon 356, the glow 358 surrounding the Mind's Eye 356 may grow larger until it pops (or other visual or audio effects may occur). The focus improvement mechanism 350 can then alert the GEM system 10 that the player is now “focused”, and can return them to game play. For example, the player can be returned to the game play frame (e.g., 502) (a spell activity in this example) to continue from where they left off.

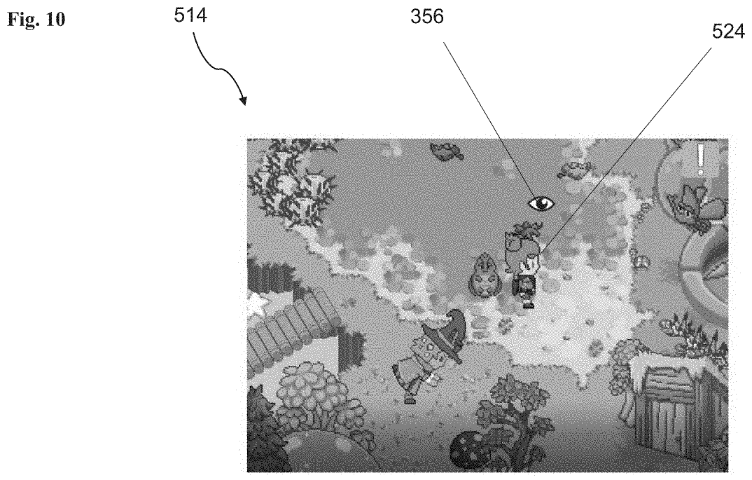

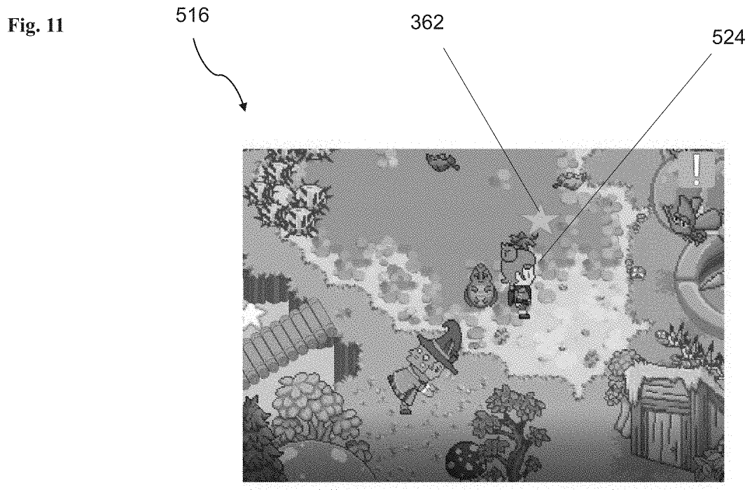

FIGS. 10-11 illustrate another example as how the focus improving mechanism 350 can be employed during game play. As shown in FIG. 10, a first full screen shot 514, the player's results in the game play can lead to the appearance of a graphical component (e.g., the Mind's Eye 356) which appears above the avatar 524 of the user. In a second full screen shot 516 (FIG. 11), a star 362 may appear above the player avatar 524, requiring the user to click the mouse pointer 360 on the star 362 to invoke the focusing technique discussed above.

In this example, the GEM system 10 might detect whether the player had answered a certain number of questions correctly, or attempted a certain number of questions and can feed that information back to the game developer. Another is that the game could try different types of animations (e.g. glowing auras or larger or smaller Mind's Eyes) and see whether one appeared more popular or helped more players progress further in the game.

In addition, the focus improving mechanism 350 can also measure the mood of the user. For example, the focus improving mechanism 350 can ask the player how they are feeling at that time, by, for instance, selecting an emotion from a list of emotions, or from a picture of a face icon displaying emotion. Some emotions might include happy, grumpy, sad, frustrated, angry, annoyed, etc. The player may be asked how they feel both before and after, for example, the Mind's Eye exercise to see whether the exercise improves their mood.

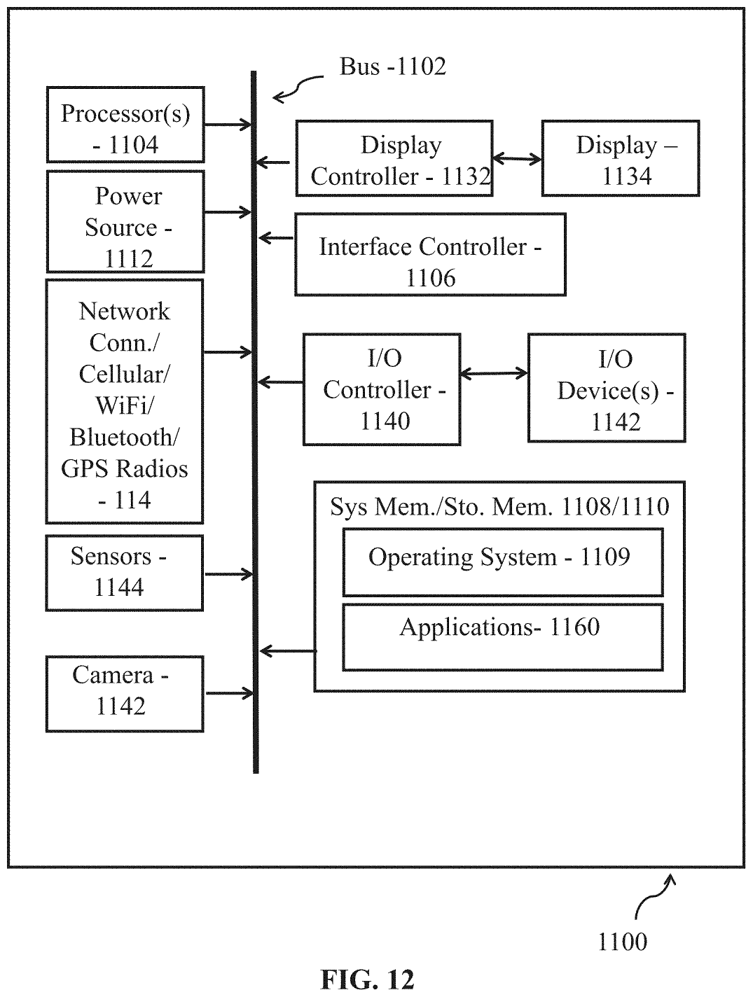

In an aspect, as discussed above, the GEM system 10 relies on user devices 100 of users (e.g., smart phones and tablets) which have certain built-in hardware (e.g., a display panel, a front facing camera, gyroscope, and accelerator) to carry out some functionality. Further, the GEM system 10 utilizes the GEM sever 300, with its built-in hardware, to carry out certain functions as well. These hardware components of both the user devices 100 and the server 300 can be found on standard mobile devices and servers, and are well known in the art. FIG. 12 is a diagram of a computing device 1100 that conveys components of both the user devices 100 and servers 300 utilized by the GEM system 10 according to an aspect of the present invention. The computing device 1100 includes a computer bus 1102 coupled to at least one or more processors 1104, one or more interface controllers 1106, system memory 1108, data storage 1110 (e.g., database 310 for the server), a power source 1112, communication means 1114, sensors 1120, user interfaces 1130, display controllers 1132, and displays 1134. The power source 1112 for the computing device 1100 may be a plug-in, battery, fuel cells, solar panels for receiving and storing solar energy, or a device for receiving and storing wireless power.

The processor 1104 can contain a plurality of processers 1104. In an aspect, the processor 104 can be a general-purpose processor, a special-purpose processor, a conventional processor, a digital signal processor, a plurality of microprocessors, a controller, a microcontroller, single core processor, a multi-core processor, an Application Specific Integrated Circuit, a Field Programmable Gate Array circuit, or any other type of integrated circuit. The system memory 1108 can also house the operating system 1109 and various applications 1160.

The display controller 1134 connects to one or more displays 1134. The one or more displays 1134 may include a touch screen display, a liquid crystal display, light emitting diode display, field emission display, organic light-emitting diode display, flexible organic light emitting diode display, or the like. Input/output (I/O) controllers 1140 and I/O devices 1142 are connected via the computer bus 1102. The I/O input devices 1142 can include, but is not limited to, side buttons, a touchscreen, a speaker, microphone, keyboard, keypad, touchpad, display, touchscreen, wireless gesture device, a digital camera, a digital video recorder, a force-feedback device, or the like. In an exemplary aspect, the I/O devices include at least a touchscreen, a front facing camera, buttons, microphones, and sensors, as discussed below.

The computing device 1100 can include a plurality of sensors 1144, such as, but not limited to, motion, proximity, light, optical, chemical, environmental, moisture, acoustic, heat, temperature, RFID, biometric, face recognition, image, photo, or voice recognition sensors and touch detectors (not shown) for detecting any touch inputs, including multi-touch inputs, for one or more display devices. One or more interface controllers 1106 may communicate with touch detectors and I/O controller 1140 for determining user inputs to the computing device 1100. The computing devices 1100 can include various communication means 1150, including, but not limited to, network connectors, and various radios (including, but not limited to, Wi-Fi, Bluetooth, GPS, Cellular, NFC, and the like), for communicating with other devices.

The system memory 1108 and storage memory 1110 may be any disk based or solid-state memory device for storing data, including volatile or non-volatile memory. The system memory 1108 and storage memory 1110 can host the operating system 1109, and also store applications 1160.

Although several aspects have been disclosed in the foregoing specification, it is understood by those skilled in the art that many modifications and other aspects will come to mind to which this disclosure pertains, having the benefit of the teaching presented in the foregoing description and associated drawings. It is thus understood that the disclosure is not limited to the specific aspects disclosed hereinabove, and that many modifications and other aspects are intended to be included within the scope of any claims that can recite the disclosed subject matter.

It should be emphasized that the above-described aspects are merely possible examples of implementations, merely set forth for a clear understanding of the principles of the present disclosure. Any process descriptions or blocks in flow diagrams should be understood as representing modules, segments, or portions of code which comprise one or more executable instructions for implementing specific logical functions or steps in the process, and alternate implementations are included in which functions may not be included or executed at all, can be executed out of order from that shown or discussed, including substantially concurrently or in reverse order, depending on the functionality involved, as would be understood by those reasonably skilled in the art of the present disclosure. Many variations and modifications can be made to the above-described aspect(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

That which is claimed is:

1. A system for evaluating gameplay of an interactive program, the system comprising:
a. a server configured to:
i. place game tags within the code of the interactive program;
ii. receive data captured by the game tags from a user device using the interactive program; and
iii. store and evaluate the data captured from the game tags to evaluate and improve the interactive program.

2. The system of claim 1, further comprising a game tag manager configured to allow a developer to place the game tags with game play of the interactive program.

3. The system of claim 1, wherein the game tags comprise special tag lines of code configured to pull data generated during use of the interactive program on the user device.

4. The system of claim 3, wherein the game tags are configured to pull data when triggered.

5. The system of claim 4, wherein the pulled data comprises a location of a game tag, a game ID, and a broadcast.

6. The system of claim 1, wherein the user device comprises a plurality of user devices, wherein the server is configured to receive, store and evaluate the data from the plurality of user devices.

7. The system of claim 6, wherein the interactive program comprises a plurality of interactive programs.

8. The system of claim 7, wherein the server is configured to evaluate each of the interactive programs.

9. The system of claim 6, further comprising an analyzer program, the analyzer program configured to apply questions to filter the data captured in order to further evaluate the interactive program.

10. The system of claim 9, wherein the analyzer program is configured to analyze the captured data without needing identification of users of the plurality of user devices.

11. The system of claim 1, further comprising an analyzer program and a reporting module, wherein the reporting module is configured to report back performance within the interactive program of a user of the user device based upon the evaluation of the data captured from the game tags.

12. The system of claim 1, further comprising a focus improving mechanism that is presented on the user device when the evaluation of the data captured from the game tags indicates a lack of focus.

13. The system of claim 12, wherein the focus improving mechanism provides audio instructions to the user.

14. The system of claim 1, wherein the server is configured to receive the data captured by the game tags in real time.

15. A system for evaluating interaction of a user with an interactive program, the system comprising:
a. a sever comprising:
i. communication means;
ii. a game tag manager configured to place game tags at events of gameplay of the interactive program;
iii. a database;
iv. an analyzer program; and
v. a reporting module;
b. a plurality of user devices, each user device comprising;
i. communication means; and
ii. the interactive program, wherein the game tags are configured to pull gameplay data captured during a user's interactions with the interactive program on the user device when the game tag is triggered, the data captured comprising a program identification, game tag location, and broadcast, wherein the game tags upon triggered call upon the communication means of the user device to send the data captured to the server,
wherein the server is configured to:
store the data captured by the game tags in the database;
provide questions, via the analyzer program, to assist in filtering the captured data to analyze the captured data to evaluate effective of the interactive program;
evaluate, via the analyzer program, an individual's performance; and
report, via the reporting module, the individual's performance.
